(19) United States
(12) Patent Application Publication
(10) Pub. No.: US 2012/0239128 A1
(43) Pub. Date: Sep. 20, 2012

(54) STENT AND DELIVERY SYSTEM

(75) Inventors: Brian Tischler, New Brighton, MN (US); James F. Hemerick, Brooklyn Park, MN (US); Daniel Gregorich, Plymouth, MN (US); Michael P. Meyer, Richfield, MN (US); Timothy J. Mickley, Corcoran, MN (US); Jan Weber, Maastricht (NL); Dennis A. Boismier, Shorewood, MN (US)

(73) Assignee: BOSTON SCIENTIFIC SCIMED, INC., Maple Grove, MN (US)

(21) Appl. No.: 13/240,678

(22) Filed: Sep. 22, 2011

Related U.S. Application Data
(60) Provisional application No. 61/453,301, filed on Mar. 16, 2011.

(51) Int. Cl. A61F 2/84; A61F 2/82

(52) U.S. Cl. 623/1.11; 623/1.16

(57) ABSTRACT

Techniques are disclosed that allow a stent to be folded within a balloon of a stent delivery system. In one example, a stent has first and second columns that extend from a proximal end to a distal end along a longitudinal axis. Each of the first columns extend substantially parallel to the longitudinal axis and comprise a plurality of elements that each have a substantially polygonal shape. Each of the second columns extend substantially parallel to the longitudinal axis and are between adjacent first columns. Each of the respective second columns include a plurality of first connectors that have a curvilinear shape and that extend in a substantially circumferential direction to connect adjacent first columns. The stent also has a plurality of second connectors, wherein at least one of the second connectors extends between a first element and a second element.

### CROSS-REFERENCE TO RELATED APPLICATIONS

This application claims the benefit of U.S. Provisional Application No. 61/453,301, entitled, “STENT AND DELIVERY SYSTEM,” by Dennis A. Boismier, James F. Hemerick, Daniel Gregorich, Michael P. Meyer, Timothy J. Mickley, Jan Weber, and Brian Tischler, and filed on Mar. 16, 2011, the entire contents of which being incorporated herein by reference.

### TECHNICAL FIELD

The disclosure relates to endoprosthesis devices for implantation within a body vessel and delivery systems for delivering such devices.

### BACKGROUND

A stent is a medical device introduced to a body lumen and is well known in the art. Typically, a stent is implanted in a blood vessel at the site of a stenosis or aneurysm endoluminally, i.e. by so-called “minimally invasive techniques” in which the stent in a radially reduced configuration, optionally restrained in a radially compressed configuration by a sheath and/or catheter, is delivered by a stent delivery system or “introducer” to the site where it is required. The introducer may enter the body from an access location outside the body, such as through the patient's skin, or by a “cut down” technique in which the entry blood vessel is exposed by minor surgical means.

Stents, grafts, stent-grafts, vena cava filters, expandable frameworks, and similar implantable medical devices are radially expandable endoprostheses which are typically intravascular implants capable of being implanted transluminally and enlarged radially after being introduced percutaneously. Stents may be implanted in a variety of body lumens or vessels such as within the vascular system, urinary tracts, bile ducts, fallopian tubes, coronary vessels, secondary vessels, etc. They may be self-expanding, expanded by an internal radial force, such as when mounted on a balloon, or a combination of self-expanding and balloon expandable (hybrid expandable).

### SUMMARY

In general, this disclosure describes techniques that allow a stent to be folded within a balloon of a stent delivery system. In particular, various techniques described throughout this disclosure may allow flexible portions of a stent to be aligned with portions of a balloon of a stent delivery system that undergo high deformation during pleating of the delivery balloon prior to folding. In this manner, the stent may be embedded within the folds of the delivery balloon during the folding process.

In one example, the disclosure is directed to a stent having a proximal end, a distal end, and a longitudinal axis. The stent comprises a plurality of first columns, the plurality of first columns extending from the proximal end to the distal end along the longitudinal axis, each of the respective first columns extending substantially parallel to the longitudinal axis, each of the respective first columns comprising a plurality of elements, each of the elements having a substantially polygonal shape. The stent further comprises a plurality of second columns, the plurality of second columns extending from the proximal end to the distal end along the longitudinal axis, each of the respective second columns extending substantially parallel to the longitudinal axis, each of the respective second columns between adjacent first columns, and each of the respective second columns comprising a plurality of first connectors, each of the first connectors having a curvilinear shape and extending in a substantially circumferential direction to connect adjacent first columns. The stent further comprises a plurality of second connectors, wherein at least one of the second connectors extends between a first element and a second element.

In another example, the disclosure is directed to a stent delivery system. The system comprises a catheter that comprises an expandable balloon, the balloon having an unpleated state and a pleated state, the balloon having at least two pleats in the pleated state, adjacent pleats being separated by a trough in the pleated state. The system further comprises a stent having a longitudinal axis, the stent comprising a plurality of interconnected annular bands. Each annular band comprises a plurality of elements, each of the elements having a substantially polygonal shape, and a plurality of first connectors, at least one of first connectors extending between adjacent elements of an annular band, at least one of the first connectors being disposed adjacent at least one pleat of the balloon, and at least another of the first connectors being disposed adjacent at least one trough of the balloon. The stent further comprises a plurality of second connectors, wherein adjacent annular bands are connected by at least one second connector.

The details of one or more aspects of the disclosure are set forth in the accompanying drawings and the description below. Other features, objects, and advantages will be apparent from the description and drawings, and from the claims.

### BRIEF DESCRIPTION OF DRAWINGS

FIG. 1 is a side view of a stent delivery system that may be used in accordance with various techniques of this disclosure.

FIGS. 2A-2B are perspective views of a balloon in an unpleated state and a pleated state, respectively, that may be used in conjunction with the stent delivery system shown in FIG. 1.

FIG. 3A is a perspective view of an example stent for use with the pleated balloon shown in FIG. 2B, in accordance with this disclosure.

FIG. 3B is an end view of the example stent and pleated balloon shown in FIG. 3A.

FIG. 3C is an end view of the example stent and pleated balloon of FIG. 3A, shown in a wrapped state.

FIG. 3D is an end view of the example stent and pleated balloon of FIG. 3A, shown in an unwrapped state.

FIG. 4A is a flat plan view of an example stent, in accordance with this disclosure.

FIG. 4B is an end view of an example balloon in a pleated state that may be aligned with the example stent of FIG. 4A.

FIGS. 5-14 are flat plan views of example elements that may form part of a stent, in accordance with various techniques of this disclosure.

FIG. 15A is a flat plan view of another example stent, in accordance with this disclosure.

FIG. 15B is an end view of an example balloon in a pleated state that may be aligned with the example stent of FIG. 15A.

FIG. 16A is a flat plan view of another example stent, in accordance with this disclosure.

FIG. 16B is an end view of an example balloon in a pleated state that may be aligned with the example stent of FIG. 16A.

FIG. 17A is a flat plan view of another example stent, in accordance with this disclosure.

FIG. 17B is an end view of an example balloon in a pleated state that may be aligned with the example stent of FIG. 17A.

FIG. 18A is a flat plan view of another example stent, in accordance with this disclosure.

FIG. 18B is an end view of an example balloon in a pleated state that may be aligned with the example stent of FIG. 18A.

FIGS. 19-25 are flat plan views of example flexible connectors that may connect elements of a stent, in accordance with this disclosure.

FIGS. 26-35 are flat plan views of example connectors that may connect adjacent annular bands of a stent, in accordance with this disclosure.

FIGS. 36-40 are flat plan views of example connector configurations that may connect adjacent annular bands of a stent, in accordance with this disclosure.

FIGS. 41A-41D are cross-sectional views of example balloons that may be used to implement various techniques of this disclosure.

FIG. 42A depicts an end view of an example stent and balloon prior to folding using certain techniques of this disclosure.

FIG. 42B depicts an end view of the example stent and balloon of FIG. 42A after folding.

FIG. 43A is a flat plan view of an unwrapped self-expanding stent held down by deformable rings.

FIG. 43B is a cross-sectional view of a self-expanding stent held down by deformable rings.

FIG. 43C is a cross-sectional view of the self-expanding stent of FIG. 43B in an expanded state.

FIG. 44 is a perspective view of another example stent for use with a pleated balloon, in accordance an example of this disclosure.

### DETAILED DESCRIPTION

This disclosure describes techniques that allow flexible portions of a stent to be aligned with portions of a balloon of a stent delivery system that undergo high deformation during pleating of the delivery balloon prior to folding. As such, using certain techniques of this disclosure, the stent can be embedded within the folds of the delivery balloon during the folding process. Embedding the stent within the folds of the balloon provides one or more advantages over designs that do not utilize such techniques. For example, embedding the stent within the folds of the balloon helps secure the stent to the balloon, reduce the effects of foreshortening, i.e., stent length changing upon stent deployment, reduce the chance that the balloon will burst at low pressures due to stent struts pinching the balloon material, and minimize the surface area of the stent in contact with blood (thereby reducing systemic loss of drug or other active pharmaceutical ingredient off the surface of the stent).

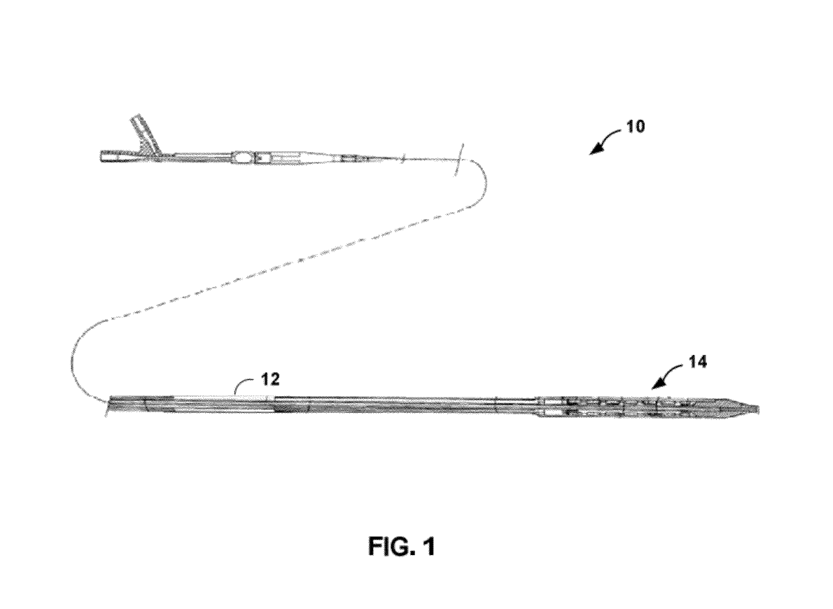

FIG. 1 is a side view of a stent delivery system that may be used in accordance with various techniques of this disclosure. As seen in FIG. 1, stent delivery system 10 includes a catheter 12 having distal end 14 upon which a delivery balloon and stent may be secured in order to deliver the stent to a site within a patient's body. Stent delivery systems such as stent delivery system 10 are well known and, for purposes of conciseness, will not be described in detail in this disclosure.

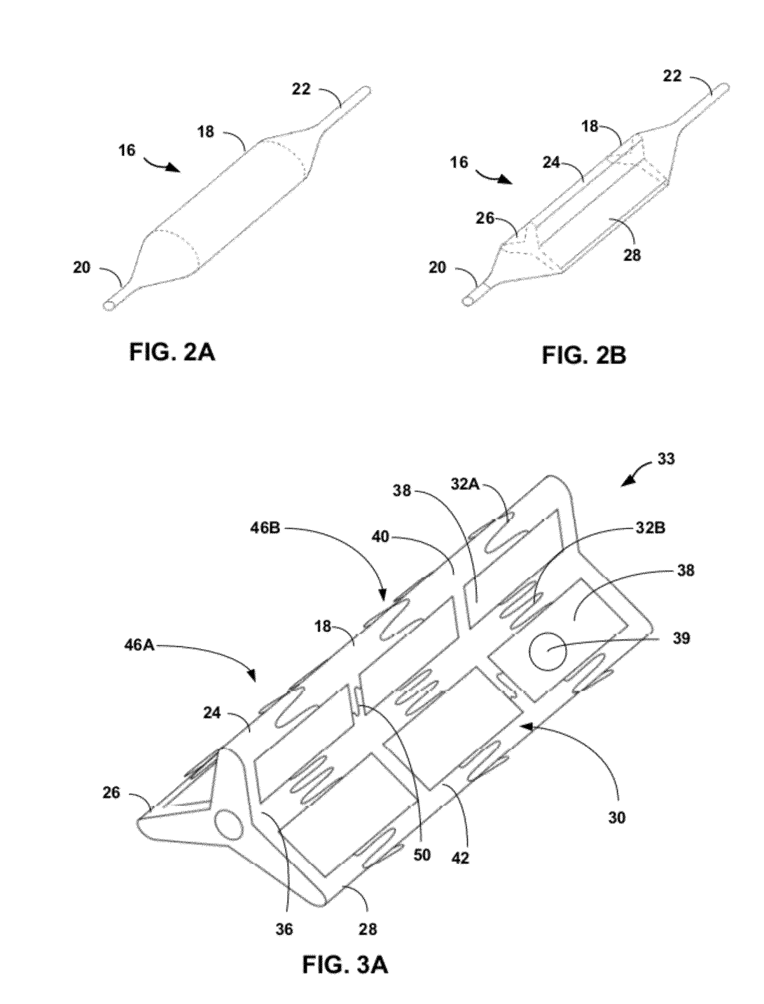

FIGS. 2A-2B are perspective views of a delivery balloon in an unpleated state and a pleated state, respectively, that can be used in conjunction with the stent delivery system shown in FIG. 1. In FIG. 2A, delivery balloon 16 with balloon body 18, shown in an unpleated, or expanded state, can be secured to distal end 14 of catheter 12 (FIG. 1), e.g., by adhesive attachment of proximal end 20 of balloon 16 to an outer shaft of catheter 12 and distal end 22 of a balloon 16 to an inner shaft of catheter 12.

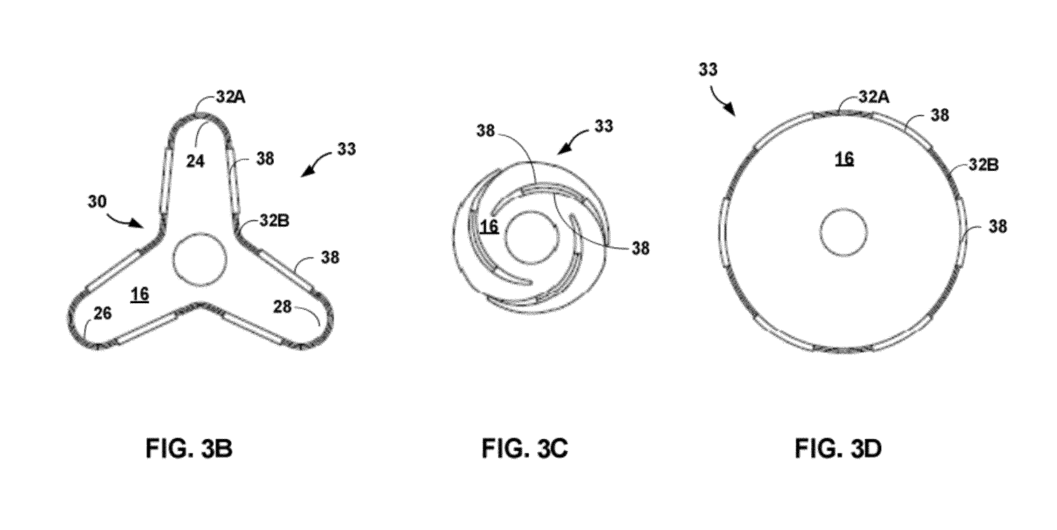

FIG. 2B depicts delivery balloon 16 with balloon body 18 in a pleated state, prior to folding of the balloon. As depicted in FIG. 2B, balloon body 18 includes three pleats, namely pleats 24, 26 and 28. In other configurations, balloon body 18 includes fewer or more pleats, as described in more detail below. In other words, the balloon body, or balloon, has at least two pleats in the pleated state. By providing pleats that can be folded, the profile of balloon body 18 is reduced, thereby allowing a stent (shown in FIG. 3A, for example) to be mounted over the balloon and delivered via a stent delivery system, e.g., stent delivery system 10 of FIG. 1, to a site within the patient's body.

FIG. 3A is a perspective view of an example stent for use with the pleated balloon shown in FIG. 2B, in accordance an example of this disclosure. In particular, FIG. 3A depicts one example stent, shown generally at 30, that includes a plurality of flexible connectors, e.g., connectors 32A, that can be disposed adjacent pleats of balloon 16, e.g., pleat 24, and flexible connectors, e.g., connectors 32B, that may disposed adjacent troughs 36 of balloon 16 that are formed between the pleats, e.g., between pleats 24 and 28. In other words, the flexible connectors, e.g., connectors 32A, 32B, are aligned with portions of balloon body 18 that highly deform upon pleating of the stent and balloon combination prior to folding. Connectors 32A, 32B are referred to generally herein as “connectors 32”. The stent and pleated balloon combination is shown generally at 33.

Stent 30 further includes a plurality of “stiff” elements 38 that can be disposed adjacent “flat” portions of pleated balloon 16, e.g., portion 40, that extend between a pleat, e.g., pleat 28, and a trough, e.g., trough 36. Similarly, a plurality of elements 38 can be disposed adjacent “flat” portion 42 that extends between pleat 24 and trough 36. Although hidden in the view depicted in FIG. 3A, additional elements 38 can be disposed between pleat 26 and trough 36 as well as pleat 28 and trough 36. Although depicted in FIG. 3A as substantially rectangular, in other example configurations, elements 38 are substantially elliptical, substantially square, solid, porous, or many other shapes or configurations, as described in more detail below.

As seen in FIG. 3A, flexible connectors 32 extend between adjacent elements 38, thereby creating a plurality of annular bands, e.g., annular bands 46A, 46B, that extend along a length of balloon body 18. In the example depicted in FIG. 3A, adjacent annular bands, e.g., annular bands 46A, 46B, are connected by one or more connectors, e.g., connector 50, that extend in a generally longitudinal direction along a longitudinal axis of stent 30.

Using the techniques of this disclosure, stent 30 can be folded with balloon 16 during a folding operation of assembly, thereby embedding stent 30 within the folds of the delivery balloon. The flexible portions of stent 30, e.g., connectors 32A, 32B, are aligned with balloon portions, e.g., pleats 24, 26, 28 and troughs 36, that undergo high deformation during pleating of the delivery balloon prior to folding. The flexible portions of stent 30, e.g., connectors 32A, 32B, permit stent 30 to bend over pleats or bend in troughs between pleats, respectively, thereby allowing the stent/balloon combination depicted in FIG. 3A, for example, to be further compressed during the folding operation, e.g., in which all pleats are folded in a clockwise or counterclockwise direction. In some example implementations, one or more elements 38 can include one or more therapeutic agent layers 39. In some configurations, therapeutic agent layers 39 are protected by an opposing pleat when in a folded state.

FIG. 3B is an end view of the example stent and pleated balloon shown in FIG. 3A. In particular, FIG. 3B depicts balloon 16 in a pleated, unwrapped state with a stent, e.g., stent 30 of FIG. 3A, disposed thereon, thereby forming stent and balloon combination 33.

FIG. 3C is an end view of the example stent and pleated balloon of FIG. 3A, shown in a wrapped state. In particular, FIG. 3C depicts balloon 16 in a pleated, wrapped state with a stent, e.g., stent 30 of FIG. 3A, disposed thereon, thereby forming stent and balloon combination 33.

FIG. 3D is an end view of the example stent and pleated balloon of FIG. 3A, shown in an unwrapped state. In particular, FIG. 3D depicts balloon 16 in an unpleated, unwrapped state with a stent, e.g., stent 30 of FIG. 3A, disposed thereon, thereby forming stent and balloon combination 33.

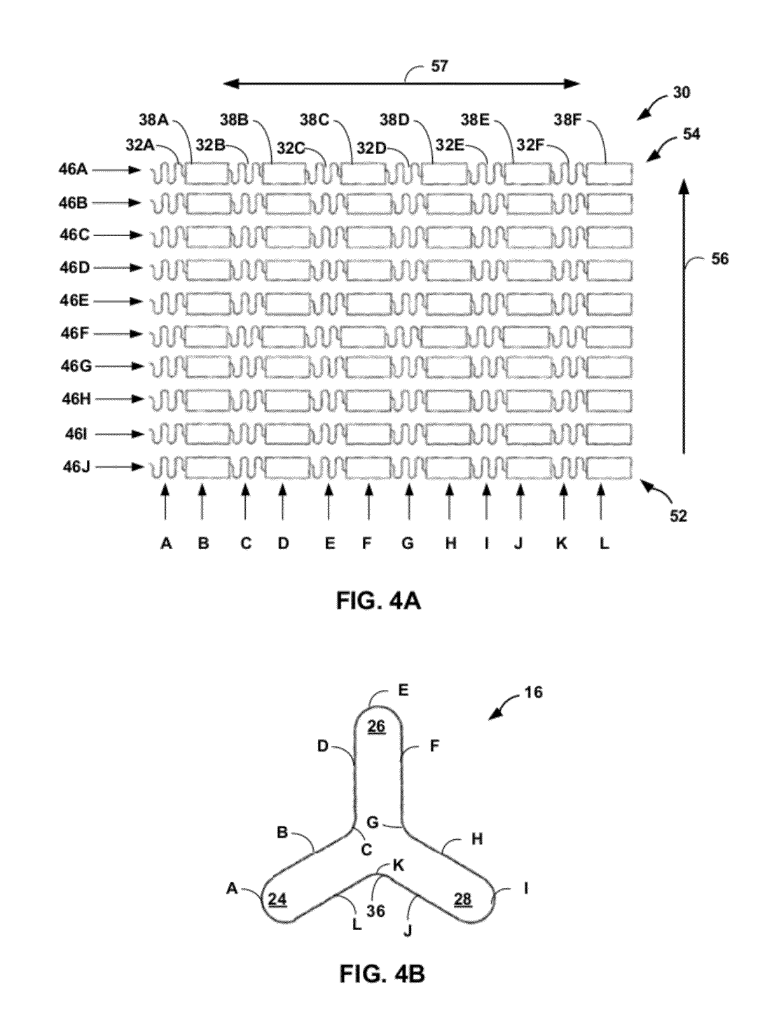

FIG. 4A is a flat plan view of an example stent, in accordance with this disclosure. The example stent of FIG. 4A, shown generally at 30, is one example stent that can be used with a balloon with three pleats, e.g., balloon 16 of FIG. 2B, in a stent delivery system, e.g., stent delivery system 10 of FIG. 1. As seen in FIG. 4A, stent 30 includes a plurality of annular bands, e.g., annular bands 46A-46J (collectively referred to in this disclosure as “annular bands 46”), that include flexible connectors 32A-32F (collectively referred to in this disclosure as “connectors 32”) that extend between stiff elements 38A-38F (collectively referred to in this disclosure as “elements 38”). Although depicted in FIG. 4A as substantially rectangular, in other example configurations, elements 38 are substantially elliptical, substantially square, or many other shapes or configurations as described in more detail below.

As seen in FIG. 4A, stent 30 includes a plurality of columns, labeled “A”-“L”, that extend from proximal end 52 of stent 30 to distal end 54 of stent 30 along longitudinal axis 56 of stent 30. For example, column “A” includes a plurality of connectors 32A, column “B” includes a plurality of elements 38A, column “C” includes a plurality of connectors 32B, and so forth.

Each of respective columns “A”-“L” of stent 30 extends substantially parallel to longitudinal axis 56. In addition, each of respective columns “A”-“L” corresponds to a portion of a balloon, as mentioned above with respect to FIG. 3A and as described in detail below with respect to FIG. 4B. For example, flexible connectors 32 of columns A, C, E, G, I, and K of stent 30 are configured to be disposed over the pleats of a balloon or within the trough between pleats of a balloon, thereby allowing stent 30 to be folded within the balloon. Generally speaking, the number of columns of flexible connectors 32 is equal to twice the number of pleats. For example, a balloon with two pleats may correspond with a stent having four flexible connector columns, and a balloon with four pleats may correspond with a stent having eight flexible connectors columns. By way of specific example, balloon 16 of FIG. 4B has three pleats and thus stent 30 of FIG. 4A has six flexible connectors columns, namely columns A, C, E, G, I, and K.

As such, in accordance with this disclosure, a stent, e.g., stent 30, includes a plurality of first columns, e.g., columns “B”, “D”, “F”, “H”, “J”, and “L”, that each extend from proximal end 52 of stent 30 to distal end 54 along longitudinal axis 56. Each of the respective first columns, e.g., columns “B”, “D”, “F”, “H”, “J”, and “L,” extend substantially parallel to longitudinal axis 56, and each of the respective first columns comprise a plurality of elements, each of the elements having a substantially polygonal shape.

In addition, in accordance with this disclosure, the stent, e.g., stent 30, includes a plurality of second columns, e.g., columns “A”, “C”, “E”, “G”, “I”, and “K”, that each extend from proximal end 52 to distal end 54 along longitudinal axis 56. Each of the respective second columns, e.g., columns “A”, “C”, “E”, “G”, “I”, and “K”, extend substantially parallel to longitudinal axis 56, and each of the respective second columns, e.g., columns “A”, “C”, “E”, “G”, “I”, and “K”, between adjacent first columns, e.g., columns “B”, “D”, “F”, “H”, “J”, and “L”. Each of the respective second columns comprise a plurality of first connectors, e.g., connectors 32F, and each of the first connectors have a curvilinear shape and extend in a substantially circumferential direction 57 to connect adjacent first columns, e.g., second column “K” connecting adjacent first columns “J” and “L”.

FIG. 4B is an end view of an example balloon in a pleated state that may be aligned with the example stent of FIG. 4A. In particular, FIG. 4B depicts balloon 16 with three pleats 24, 26, 28 that can be aligned with a stent, e.g., stent 30 of FIG. 4A, in accordance with certain techniques of this disclosure. As shown in FIG. 4B, balloon 16 further includes troughs between pleats, e.g., trough 36 between pleats 24, 28, as well as “flat” portions between adjacent pleats and troughs, e.g., flat portion “L” between pleat 24 and trough 36. It should be noted that the “flat” portions of balloon 16 are referred to as such for simplicity and are not necessarily flat. Rather, the flat portions of balloon 16, e.g., portions labeled B, D, F, H, J, and L are flat relative to the highly deformed pleat and trough regions of stent 30.

As indicated above, stent 30 of FIG. 4A includes a plurality of columns “A”-“L” that can be aligned with a corresponding portion of a balloon, e.g., balloon 16 of FIG. 4B, so that stent 30 can be embedded within balloon 16 during folding. In particular, columns “A”-“L” of stent 30 of FIG. 4A can be aligned with corresponding portions “A”-“L” of balloon 16. By way of specific example, connectors 32A of column “A” of FIG. 4A can be aligned with pleat 24, or portion “A”, of balloon 16 in FIG. 4B, elements 38A of column “B” of FIG. 4A can be aligned with flat portion “B” of balloon 16 in FIG. 4B, connectors 32B of column “C” of FIG. 4A can be aligned with a trough, or portion “C”, of balloon 16 in FIG. 4B, and so forth such that each of respective columns “A”-“L” of stent 30 of FIG. 4A is aligned with a corresponding portion “A”-“L” of balloon 16 in FIG. 4B. Thus, flexible connectors 32 of stent 30 can be aligned with portions of balloon body 18 of high deformation, e.g., pleat 24 and trough 36, and elements 38 of stent 30 can be aligned with flat portions of balloon 16, such as portions B, D, F, H, J, and L of balloon 16 in FIG. 4B.

As mentioned above, elements 38 need not be substantially rectangular. Rather, in other example configurations, stent 30 includes example elements that are substantially elliptical, substantially square, or many other shapes or engineered configurations having specific geometries.

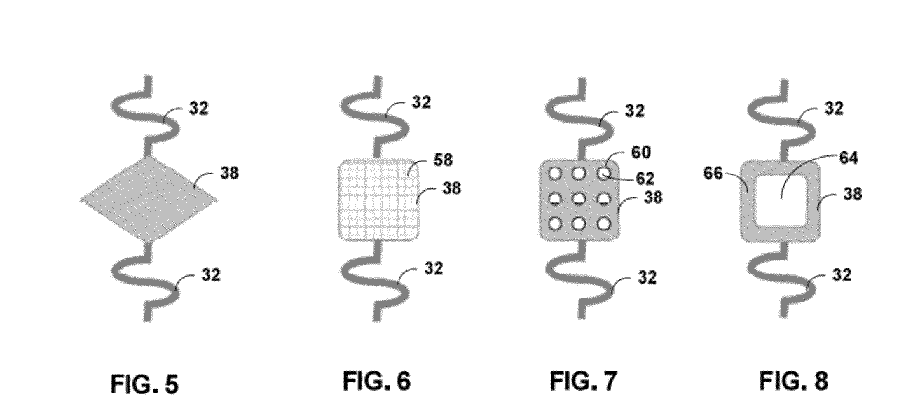

FIGS. 5-14 are flat plan views of example elements 38 that may form part of stent, e.g., stent 30, in accordance with various techniques of this disclosure. In FIG. 5, element 38 is depicted as having a substantially rhombus shape, but in other examples, can be more generally a parallelogram. Element 38 in FIG. 5 is considered solid because the material of element 38 does not define any holes or slits that extend completely through the material.

In FIG. 6, element 38 is depicted as having a substantially square shape. Element 38 in FIG. 6, unlike element 38 of FIG. 5, is not solid. Rather element 38 of FIG. 6 is porous in that the material of element 38 defines a plurality of holes 58. In this manner, element 38 of FIG. 6 can be considered similar to a screen in construction.

In FIG. 7, element 38 is depicted as having a substantially square shape that includes one or more well regions 60 that each define a well 62. Each well 62 may be configured to include one or more therapeutic agents, thereby allowing stent 30 to deliver the therapeutic agent(s) to a site within the patient's body.

In FIG. 8, element 38 is depicted as having a substantially square shape that defines a hollow region, shown generally at 64. The width of the material of element 38, shown generally at 66, can be increased or decreased, depending on the characteristics desired for element 66. For example, increasing the width of the material decreases the flexibility of element 38.

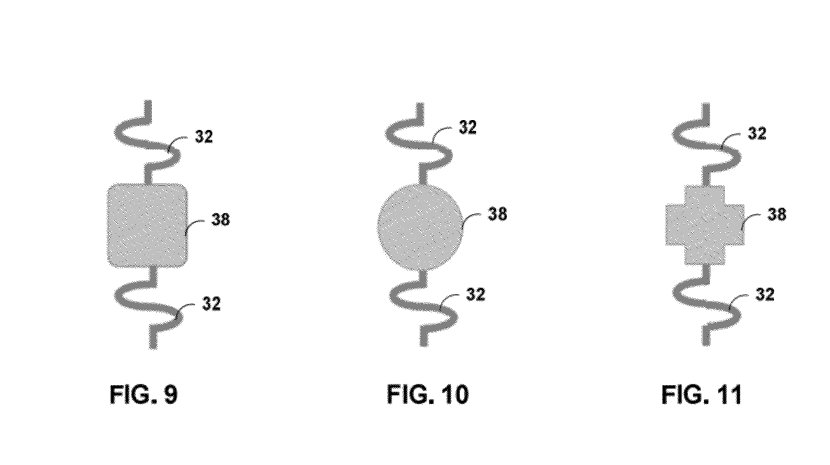

In FIG. 9, element 38 is depicted as having a substantially square shape. Unlike the example shown in FIG. 7, element 38 of FIG. 9 is solid. Although depicted as substantially square, in other example configurations, element 38 of FIG. 9 can be more generally a rectangle.

In FIG. 10, element 38 is depicted as having a substantially elliptical shape. In other example configurations, element 38 can be substantially circular in shape. Element 38 of FIG. 10 is solid.

In FIG. 11, element 38 is depicted as having a cross-like shape. Element 38 of FIG. 11 is solid.

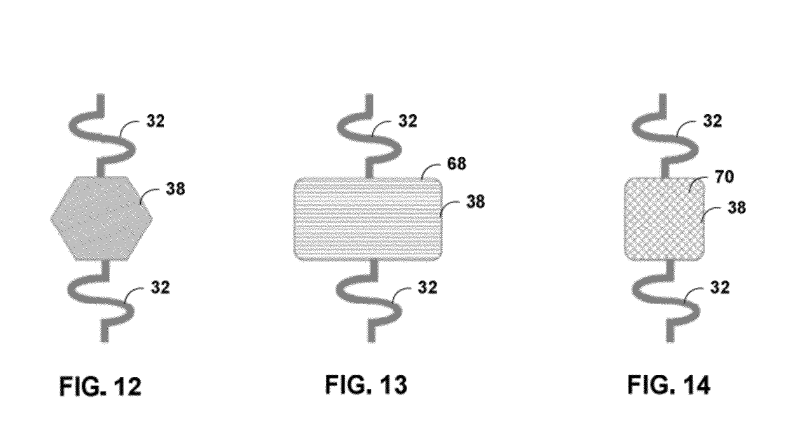

In FIG. 12, element 38 is depicted as having a substantially hexagonal shape. Element 38 of FIG. 12 is solid.

In FIG. 13, element 38 is depicted as having a substantially rectangular shape. Element 38 of FIG. 13 is porous in that the material of element 38 defines a plurality of slits 68 that extend completely through the material.

In FIG. 14, element 38 is depicted as having a substantially square shape. Element 38 of FIG. 14 is porous in that the material of element 38 defines a plurality of holes 70 that extend completely through the material. For example, the material of element 38 may form a cross-hatch pattern, as shown in FIG. 14.

It should be noted that only some of the possible configurations of elements 38 were described above. For example, any of the shapes described above as solid can, in some example configurations, be constructed to be porous, and any of the shapes described above as porous can, in other example configurations, be constructed to be solid. By way of specific example, the material of element 38 of FIG. 5 can be constructed to be porous by constructing the material to defining one or more holes, slits, or the like. In addition, while some polygonal shapes were described above, e.g., rectangle, square, hexagonal, many other polygonal shapes that are not described are within the scope of this disclosure.

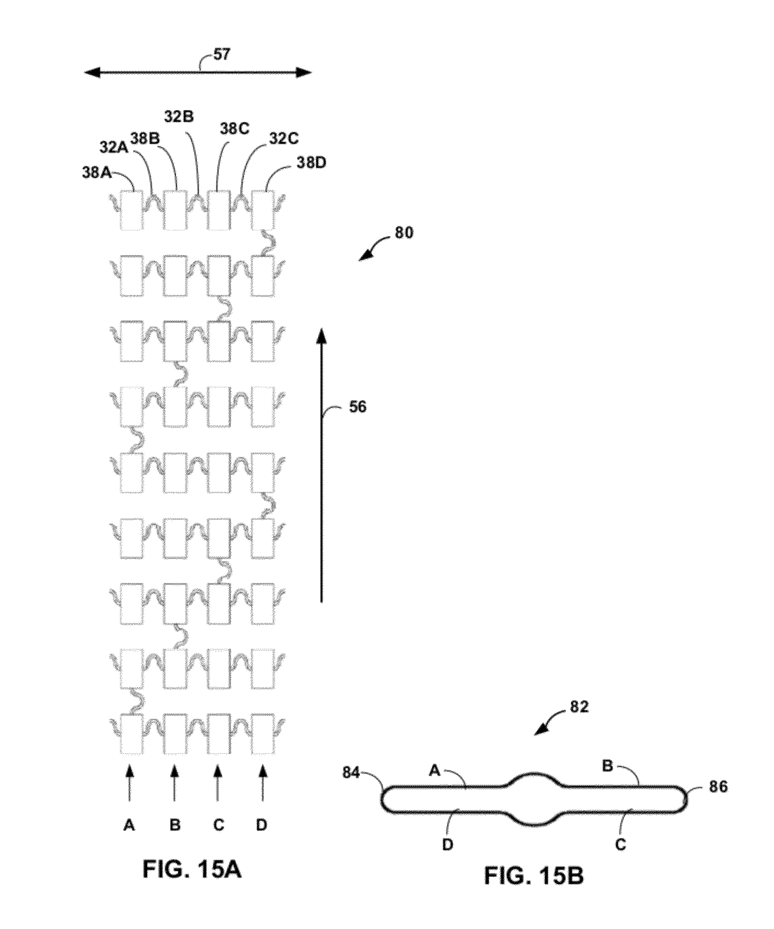

It should also be noted that although various techniques of this disclosure were described above with reference to a three-pleated balloon, e.g., balloon 16 of FIGS. 3 and 4B, the disclosure is not so limited. Rather, the techniques of this disclosure are applicable to any balloon with two or more pleats, as described in more detail below with respect to FIGS. 15A-18B.

FIG. 15A is a flat plan view of another example stent, in accordance with this disclosure. The example stent of FIG. 15A, shown generally at 80, is one example stent that can be used with a balloon with two pleats, e.g., balloon 82 of FIG. 15B, in a stent delivery system, e.g., stent delivery system 10 of FIG. 1.

As seen in FIG. 15A, stent 80 includes a plurality of annular bands, with each annular band including a plurality of flexible connectors, e.g., connectors 32A-32C, that extend between elements, e.g., elements 38A-38D. Like stent 30 described above with respect to FIG. 4A, stent 80 of FIG. 15A includes a plurality of columns, labeled “A”-“D”. It should be noted that, for purposes of conciseness, only columns that include elements 38 have been labeled. Each of respective columns “A”-“D” of stent 80 extends substantially parallel to longitudinal axis 56. In addition, each of respective columns “A”-“D” corresponds to a portion of a two-pleat balloon, described in detail below with respect to FIG. 15B.

FIG. 15B is an end view of an example balloon in a pleated state that may be aligned with the example stent of FIG. 15A. In particular, FIG. 15B depicts balloon 82 with two pleats 84, 86 that may be aligned with a stent, e.g., stent 80 of FIG. 15A, in accordance with certain techniques of this disclosure. As shown in FIG. 15B, balloon 82 further includes “flat” portions “A”-“D” that can be aligned with a corresponding column of a stent, e.g., stent 80 of FIG. 15A, so that stent 80 can be embedded within balloon 82 during folding. Alignment techniques were described in detail above with respect to FIGS. 15A-15B and, for purposes of conciseness, will not be described again.

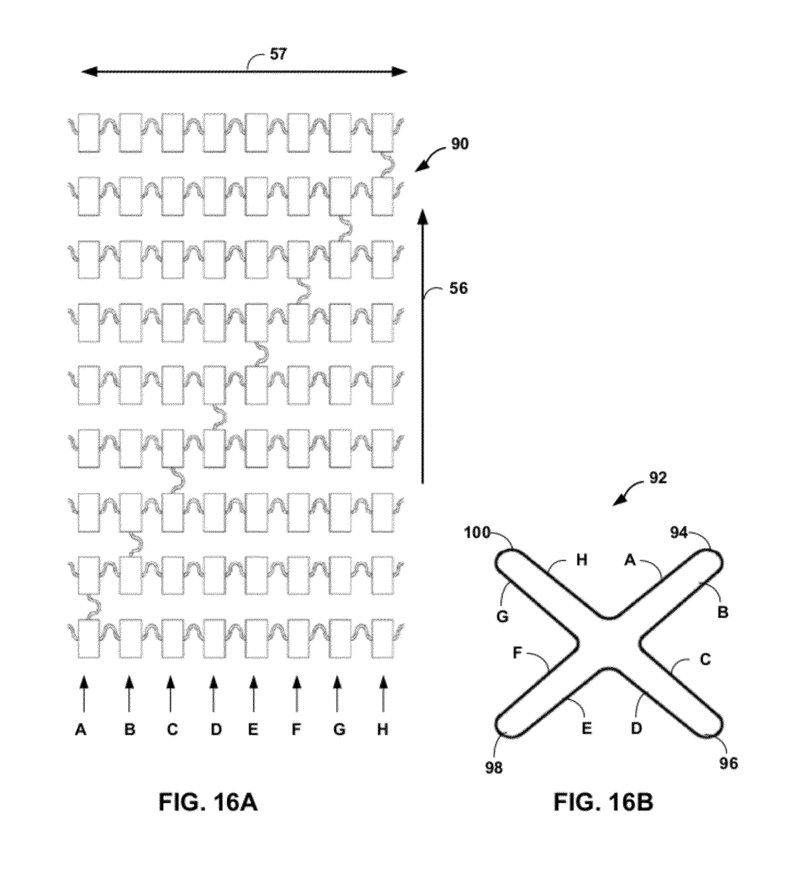

FIG. 16A is a flat plan view of another example stent, in accordance with this disclosure. The example stent of FIG. 16A, shown generally at 90, is one example stent that can be used with a balloon with four pleats, e.g., balloon 92 of FIG. 16B, in a stent delivery system, e.g., stent delivery system 10 of FIG. 1.

FIG. 16B is an end view of an example balloon in a pleated state that can be aligned with the example stent of FIG. 16A. In particular, FIG. 16B depicts balloon 92 with four pleats, namely pleats 94-100, that can be aligned with a stent, e.g., stent 90 of FIG. 16A, in accordance with certain techniques of this disclosure. Balloon 92 further includes “flat” portions “A”-“H” that can be aligned with a corresponding column of a stent, e.g., columns “A”-“H” of stent 90 of FIG. 16A, so that stent 90 can be embedded within balloon 92 during folding. Alignment techniques were described in detail above with respect to FIGS. 15A-15B and, for purposes of conciseness, will not be described again.

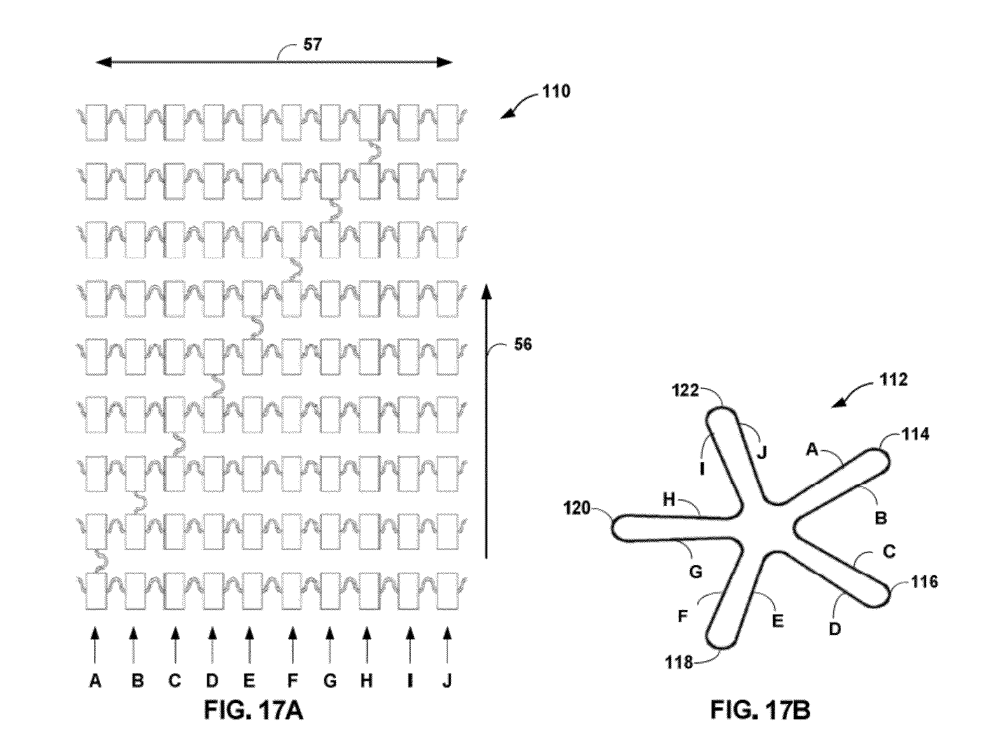

FIG. 17A is a flat plan view of another example stent, in accordance with this disclosure. The example stent of FIG. 17A, shown generally at 110, is one example stent that can be used with a balloon with five pleats, e.g., balloon 112 of FIG. 17B, in a stent delivery system, e.g., stent delivery system 10 of FIG. 1.

FIG. 17B is an end view of an example balloon in a pleated state that can be aligned with the example stent of FIG. 17A. In particular, FIG. 17B depicts balloon 112 with five pleats, namely pleats 114-122, can be aligned with a stent, e.g., stent 110 of FIG. 17A, in accordance with certain techniques of this disclosure. Balloon 112 further includes “flat” portions “A”-“J” that can be aligned with a corresponding column of a stent, e.g., columns “A”-“J” of stent 110 of FIG. 17A, so that stent 110 can be embedded within balloon 112 during folding. Alignment techniques were described in detail above with respect to FIGS. 15A-15B and, for purposes of conciseness, will not be described again.

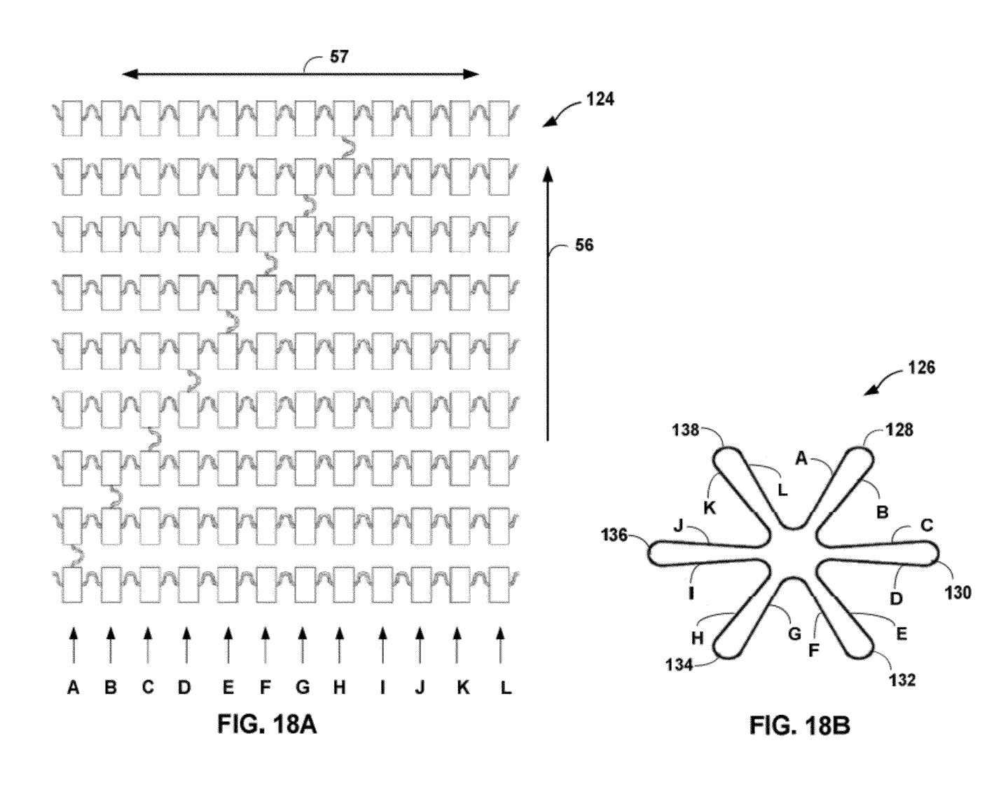

FIG. 18A is a flat plan view of another example stent, in accordance with this disclosure. The example stent of FIG. 18A, shown generally at 124, is one example stent that can be used with a balloon with six pleats, e.g., balloon 126 of FIG. 18B, in a stent delivery system, e.g., stent delivery system 10 of FIG. 1.

FIG. 18B is an end view of an example balloon in a pleated state that may be aligned with the example stent of FIG. 18A. In particular, FIG. 18B depicts balloon 126 with six pleats, namely pleats 128-138, that can be aligned with a stent, e.g., stent 124 of FIG. 18A, in accordance with certain techniques of this disclosure. Balloon 124 further includes “flat” portions “A”-“L” that can be aligned with a corresponding column of a stent, e.g., columns “A”-“L” of stent 124 of FIG. 18A, so that stent 124 may be embedded within balloon 126 during folding. Alignment techniques were described in detail above with respect to FIGS. 15A-15B and, for purposes of conciseness, will not be described again.

FIGS. 19-25 are flat plan views of example flexible connectors 32 that can connect elements 38 of a stent, e.g., stents 30, 80, 90, 110, and 124, in accordance with this disclosure. Generally speaking, connectors 32 are substantially straight or substantially curvilinear. However, substantially straight connectors 32 can include tapers, hinges, or the like in order to permit the connector to bend in or out of a plane.

In FIG. 19, connectors 32 are substantially curvilinear. In particular, FIG. 19 depicts one example of an S-shaped connector. In FIG. 20, connectors 32 are substantially curvilinear. In particular, FIG. 20 depicts U-shaped connectors.

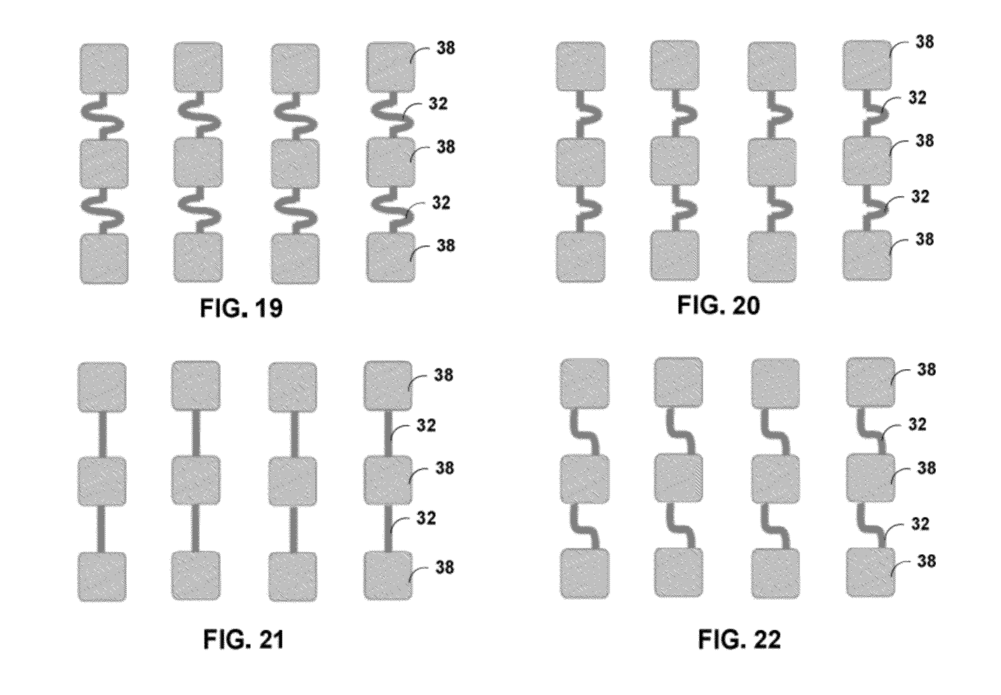

In FIG. 21, connectors 32 are substantially straight, i.e., connectors 32 do not include a curvature. In FIG. 22, connectors 32 are substantially curvilinear. In particular, FIG. 22 depicts another example of S-shaped connectors.

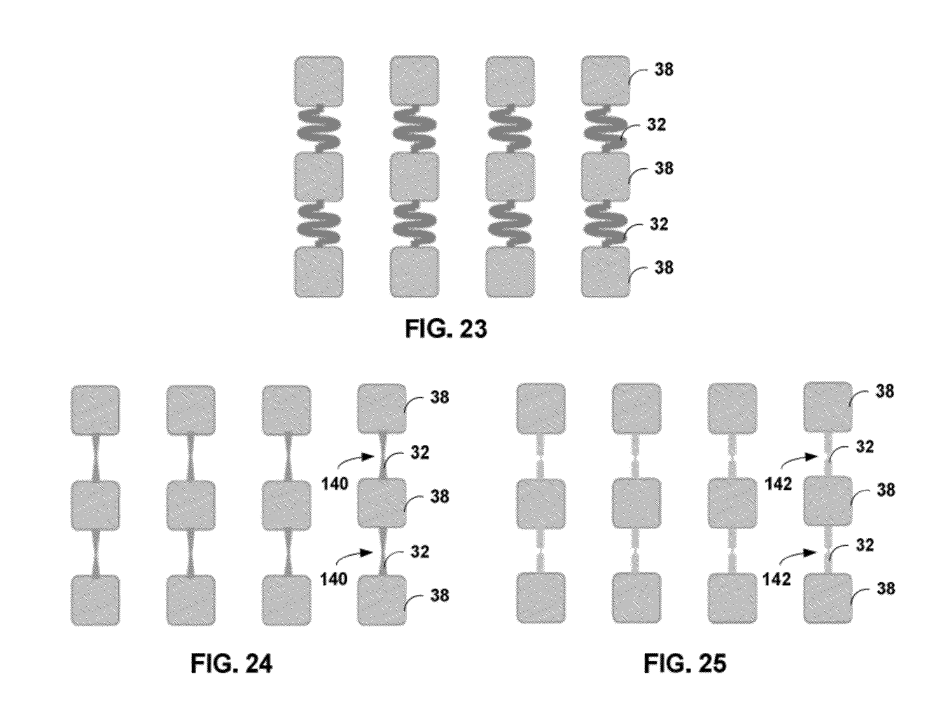

In FIG. 23, connectors 32 are substantially curvilinear. In particular, FIG. 23 depicts one example of double S-shaped connectors.

In FIG. 24, connectors 32 are substantially straight, i.e., connectors 32 do not include a curvature. In addition, connectors 32 include a tapered region, shown generally at 140, which allow connectors 32 to bend in or out of a plane. Similarly, in FIG. 25, connectors 32 are substantially straight and include a hinge region, shown generally at 142, which also allow connectors 32 to bend in or out of a plane. In some examples, hinge regions 142 are formed by removing a portion of the connector wall thickness.

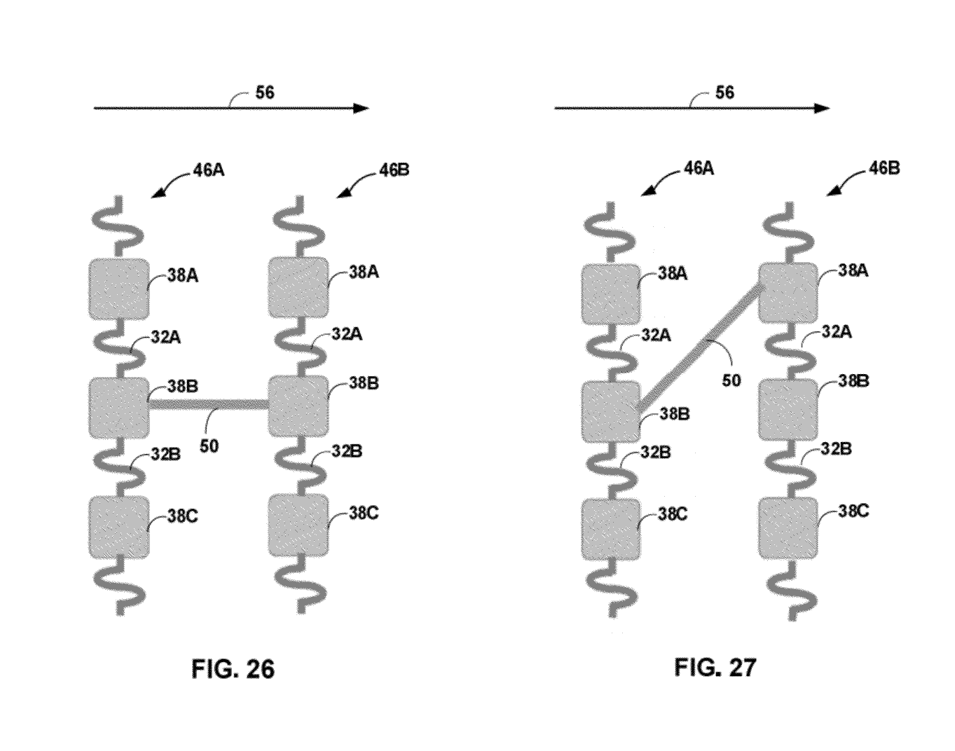

FIGS. 26-35 are flat plan views of example connectors 50 that may connect adjacent annular bands 46 of a stent, in accordance with this disclosure. Generally speaking, connectors 50 are substantially straight or substantially curvilinear.

FIG. 26 depicts two portions of two adjacent annular bands, namely bands 46A, 46B. As seen in FIG. 26, adjacent annular bands 46A, 46B are connected by connector 50, which extends in a generally longitudinal direction along longitudinal axis 56 of a stent, e.g., stents 30, 80, 90, 110, and 124. Connector 50 is substantially straight, i.e., connector 50 does not include a curvature. In the example depicted in FIG. 26, connector 50 extends between element 38B of band 46A and element 38B of band 46B. As seen in FIG. 26, element 38B of band 46A and element 38B of band 46B are longitudinally aligned along longitudinal axis 56.

In other example configurations, connector 50 extends between elements of adjacent bands 46 that are not longitudinally aligned along longitudinal axis 56, as seen in FIG. 27. In FIG. 27, connector 50 is substantially straight and extends longitudinally between element 38B of band 46A and element 38A of band 46B. Of course, other configurations are possible. For example, in some configurations, connector 50 extends between element 38B of band 46A and element 38C of band 46B. In other example configurations, connector 50 extends between element 38A of band 46A and element 38C of band 46B. In one example configuration, connector 50 extends between element 38C of band 46A and element 38A of band 46B.

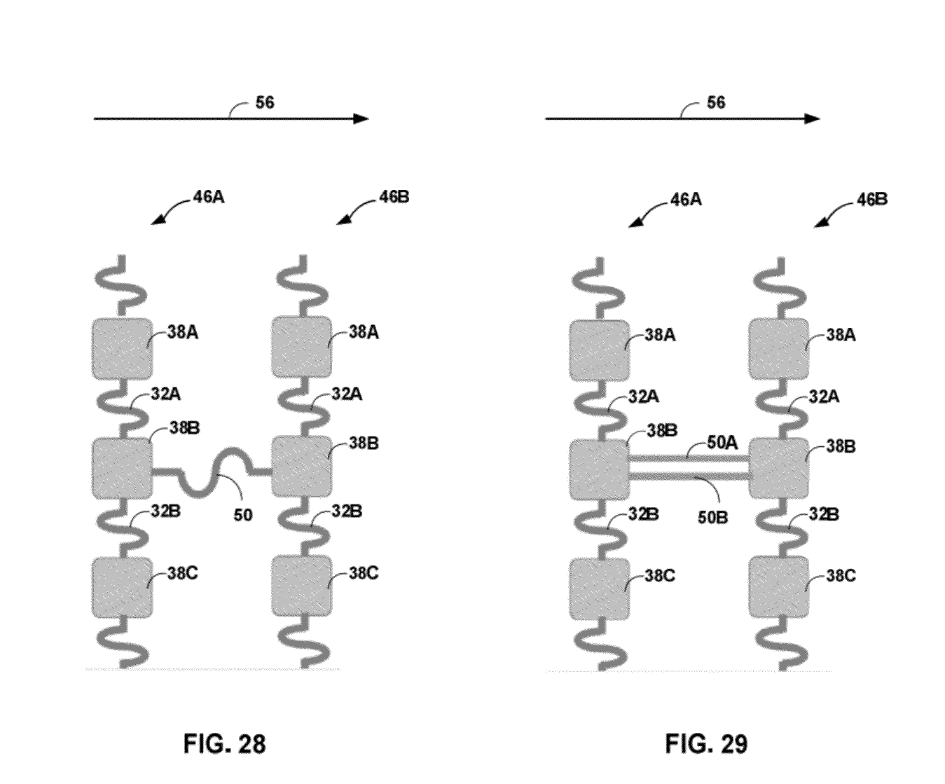

FIG. 28 depicts two portions of two adjacent annular bands, namely bands 46A, 46B connected by a substantially curvilinear connector 50 that extends in a generally longitudinal direction along longitudinal axis 56 of a stent, e.g., stents 30, 80, 90, 110, and 124. Connector 50 is one example of an S-shaped connector. As indicated above, other example S-shaped connectors can be used and are considered within the scope of this disclosure.

In the example depicted in FIG. 28, connector 50 extends between element 38B of band 46A and element 38B of band 46B. As seen in FIG. 28, element 38B of band 46A and element 38B of band 46B are longitudinally aligned along longitudinal axis 56. In other example configurations, connector 50 extends between elements of adjacent bands 46 that are not longitudinally aligned along longitudinal axis 56, as shown and described above with respect to FIG. 27.

The configuration depicted in FIG. 29 is similar to the configuration shown and described above with respect to FIG. 26, with the addition of another connector between element 38B of annular band 46A and element 38B of annular band 46B. In particular, a pair of connectors 50A, 50B extends between adjacent annular bands 46A, 46B in FIG. 29. Connectors 50A, 50B extend in a generally longitudinal direction along longitudinal axis 56 of a stent, e.g., stents 30, 80, 90, 110, and 124, and are longitudinally aligned along longitudinal axis 56. In other configurations, connectors 50A, 50B are not longitudinally aligned along longitudinal axis 56. For example, connectors 50A, 50B can extend between element 38B of band 46A and element 38C of band 46B. As another example, connectors 50A, 50B can extend between element 38A of band 46A and element 38C of band 46B. As another example, connectors 50A, 50B can extend between element 38C of band 46A and element 38A of band 46B.

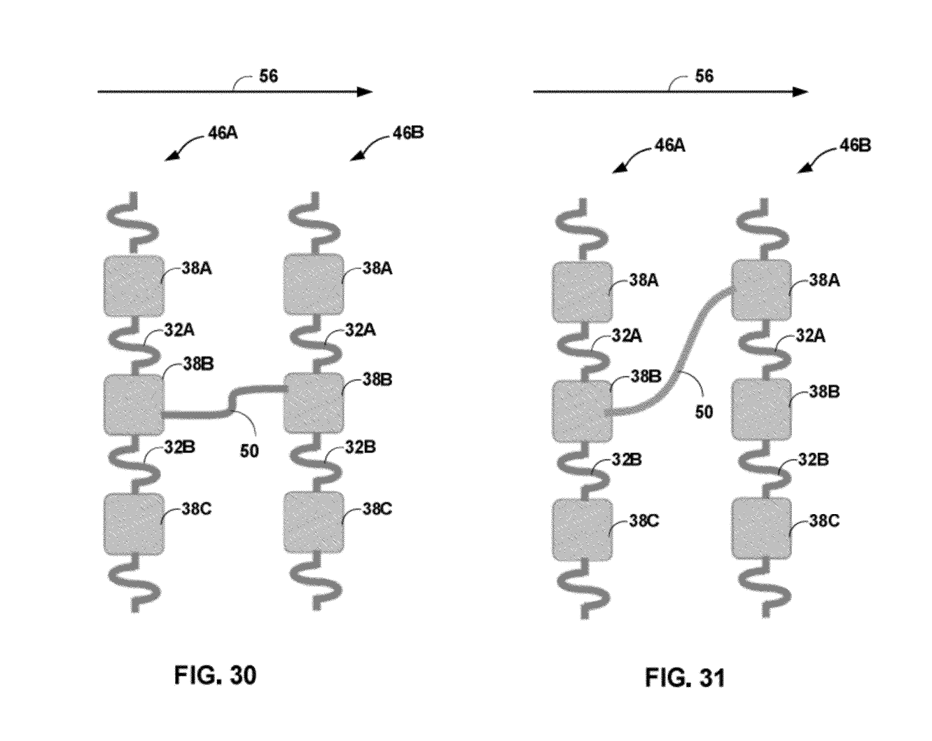

The configuration shown in FIG. 30 is similar to that shown and described above with respect to FIG. 28, with connector 50 in FIG. 30 being substantially curvilinear and another example of an S-shaped connector.

In the example depicted in FIG. 31, connector 50 extends between two elements that are not longitudinally aligned along longitudinal axis 56, namely element 38B of band 46A and element 38A of band 46B. Connector 50 in FIG. 30 is substantially curvilinear and is another example of an S-shaped connector.

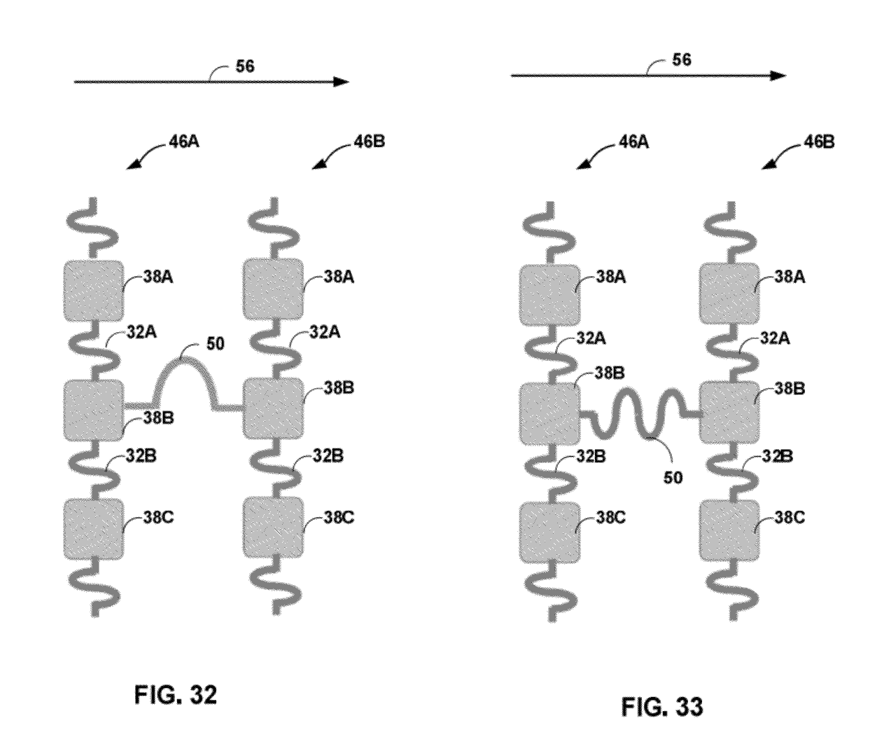

In the example configuration of FIG. 32, connector 50 is substantially curvilinear and, in particular, is U-shaped. Connector 50 extends between two elements that are longitudinally aligned along longitudinal axis 56, namely element 38B of band 46A and element 38B of band 46B, but in other configurations can extend between elements 38 that are not longitudinally aligned along longitudinal axis 56, as described above.

In the example configuration of FIG. 33, connector 50 is substantially curvilinear and, in particular, is double S-shaped. Connector 50 extends between two elements that are longitudinally aligned along longitudinal axis 56, namely element 38B of band 46A and element 38B of band 46B, but in other configurations may extend between elements 38 that are not longitudinally aligned along longitudinal axis 56, as described above.

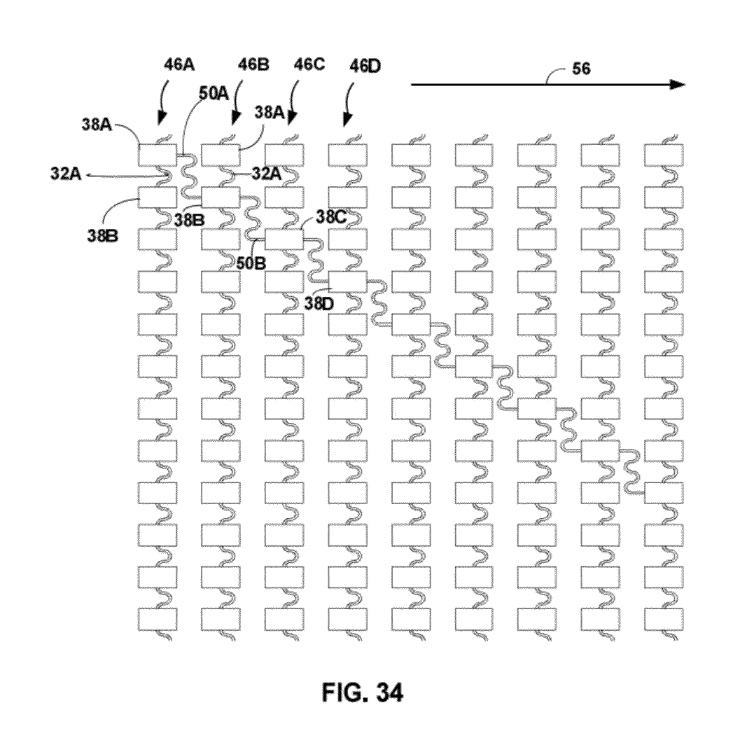

In the example depicted in FIG. 34, connectors 50 extend between two elements that are not longitudinally aligned along longitudinal axis 56. For example, connector 50A extends between element 38A of band 46A and element 38B of band 46B, connector 50B extends between element 38B of band 46B and element 38C of band 46C, and so forth. Connectors 50 in FIG. 34 are substantially curvilinear and are another example of an S-shaped connector.

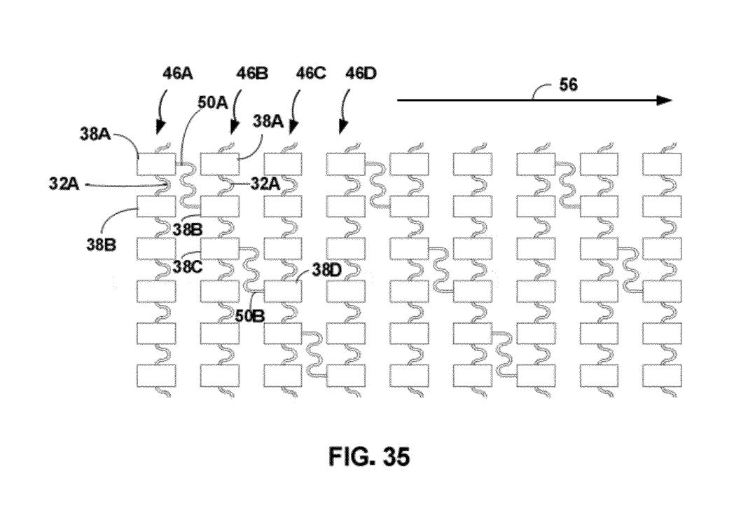

In the example depicted in FIG. 35, connectors 50 extend between two elements that are not longitudinally aligned along longitudinal axis 56. For example, connector 50A extends between element 38A of band 46A and element 38B of band 46B, connector 50B extends between element 38C of band 46B and element 38D of band 46C, and so forth. Connectors 50 in FIG. 35 are substantially curvilinear and are another example of an S-shaped connector.

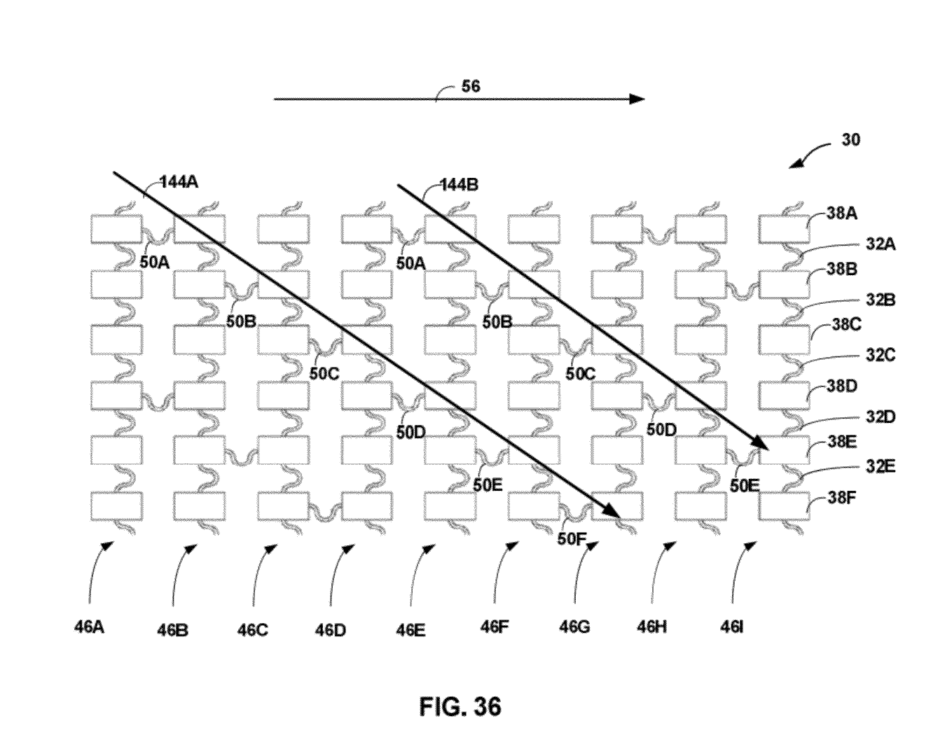

FIGS. 36-40 are flat plan views of example connector configurations that may connect adjacent annular bands of a stent, in accordance with this disclosure. FIG. 36 depicts an example stent, e.g., stent 30, that includes a plurality of connectors 50 that are arranged to form first and second substantially helical pathways, e.g., pathways 144A, 144B along longitudinal axis 56 of stent 30. For example, connectors 50A-50F are arranged to form first substantially helical pathway 144A, and connectors 50A-50E are arranged to form second substantially helical pathway 144B.

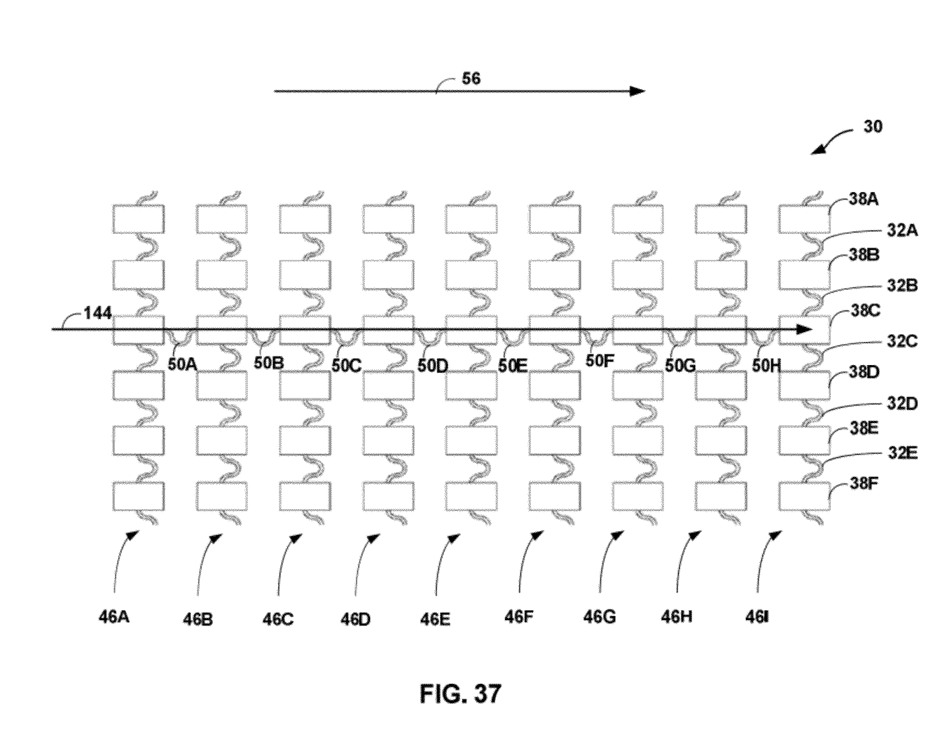

FIG. 37 depicts an example stent, e.g., stent 30, that includes a plurality of connectors 50A-50H that are arranged to form at least one pathway, e.g., pathway 144, that is substantially parallel to longitudinal axis 56 of stent 30.

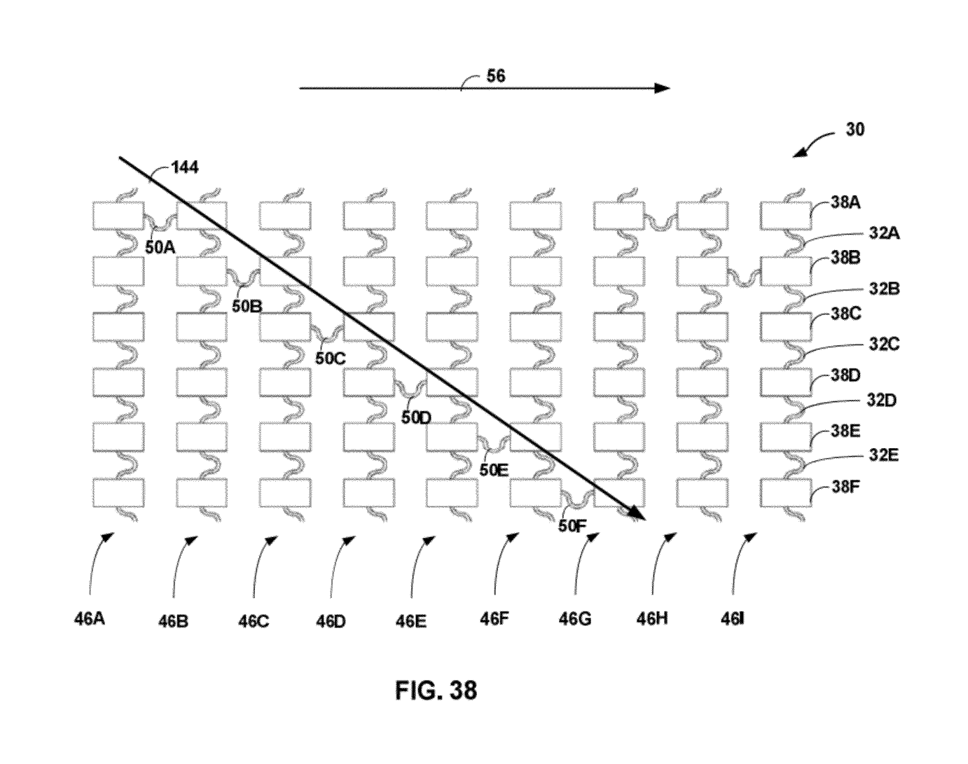

FIG. 38 depicts an example stent, e.g., stent 30, that includes a plurality of connectors 50A-50F that are arranged to form a single substantially helical pathway, e.g., pathway 144, along longitudinal axis 56 of stent 30.

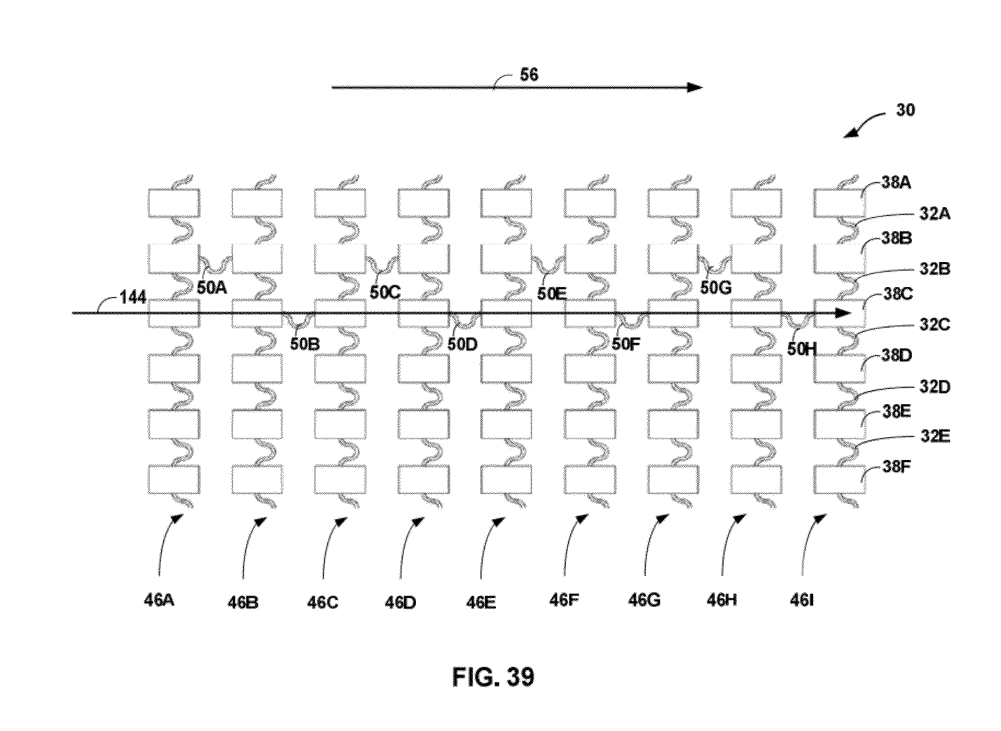

FIG. 39 depicts an example stent, e.g., stent 30, that includes a plurality of connectors 50A-50H that are arranged to form a single staggered pathway, e.g., pathway 144, along longitudinal axis 56 of stent 30.

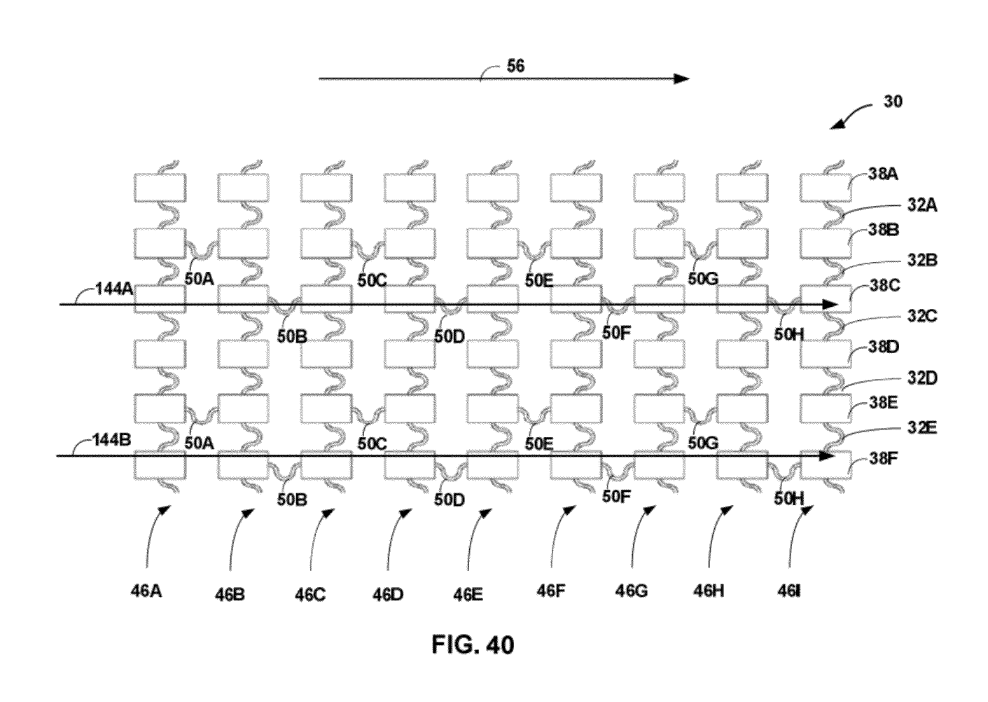

FIG. 40 depicts an example stent, e.g., stent 30, that includes a plurality of connectors 50A-50H that are arranged to form first and second staggered pathways, e.g., pathways 144A, 144B, along longitudinal axis 56 of stent 30. Although described above with respect to stent 30, pathways 144 may be used with any stent configuration, in accordance with this disclosure.

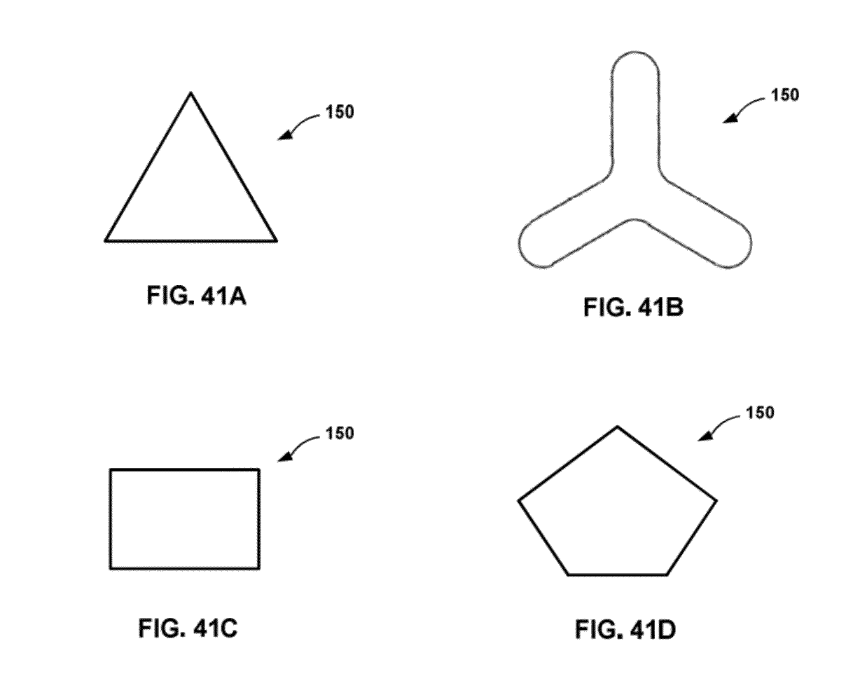

FIGS. 41A-41D are cross-sectional views of example balloons that may be used to implement various techniques of this disclosure. The techniques described above can be used in conjunction with a balloon, e.g., balloon 16, made of tubing which is a substantially circular when viewed in cross-section. In accordance with this disclosure, the balloon can be constructed from tubing that is non-circular when viewed in cross-section, as seen in FIGS. 41A-41D. In some examples, the tubing has a substantially triangular cross-sectional shape, as shown generally at 150 in FIG. 41A. In one example, the tubing has a cross-sectional shape that resembles two or more pleats, as shown generally at 150 in FIG. 41B. In some examples, the tubing has a substantially rectangular cross-sectional shape, as shown generally at 150 in FIG. 41C. In another example, the tubing has a substantially pentagonal shape, as shown generally at 150 in FIG. 41D. The shapes depicted in FIGS. 41A and 41B allows for folding of the balloon into three pleats, the shape depicted in FIG. 41C allows for folding into four pleats, and the shape depicted in FIG. 41D allows for folding into five pleats. Of course, many other cross-sections 150 can be used to implement various techniques described above, all of which are within the scope of this disclosure.

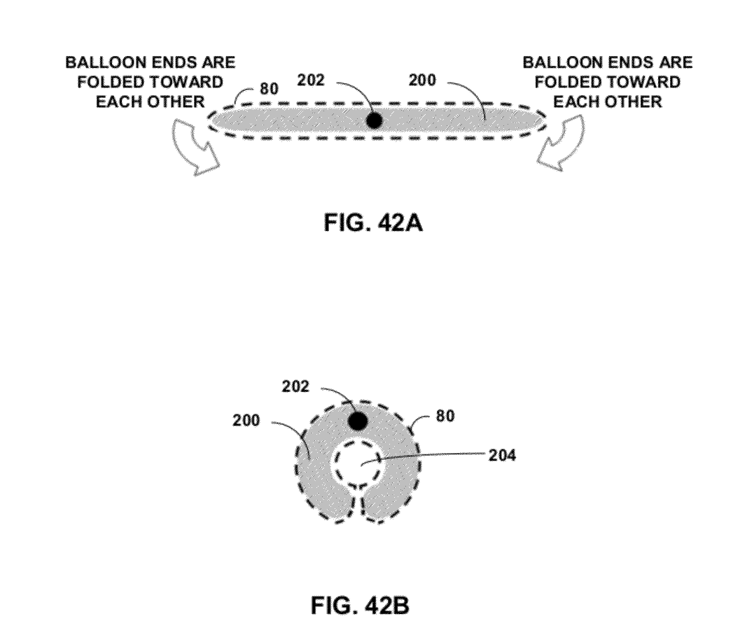

In addition to the techniques described above, this disclosure is also directed to folding techniques that can bias a stent to one side of a catheter, e.g., catheter 12 of FIG. 1, of a stent delivery system, e.g., stent delivery system 10. For example, stent 80 shown in FIG. 15A may be disposed about a balloon, e.g., a balloon having a substantially circular cross-section, as shown and described below with respect to FIGS. 42A and 42B.

FIG. 42A depicts an end view of an example stent and balloon prior to folding using certain techniques of this disclosure. In the example shown in FIG. 42A, stent 80 and balloon 200, disposed about catheter lumen 202, are flattened such that balloon 200 includes two pleats, and then the two pleats are rolled toward one another to create a “C”-shaped combination of stent and balloon, as shown in FIG. 42B. Such a stent and balloon combination provides a stent delivery system with a stent that is biased toward one side of the system. It should noted that in one example implementation, an inner space defined by folded balloon 202, shown at 204, can be used as a guidewire channel.

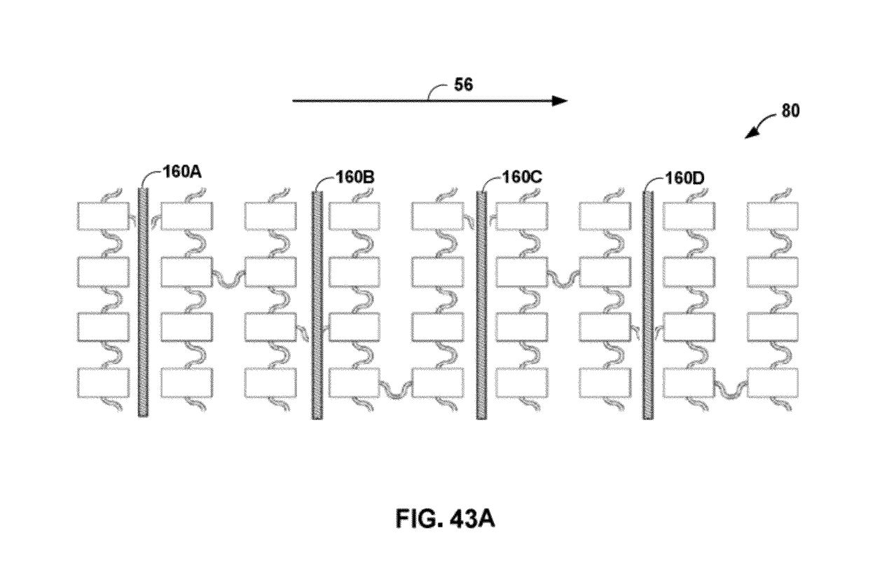

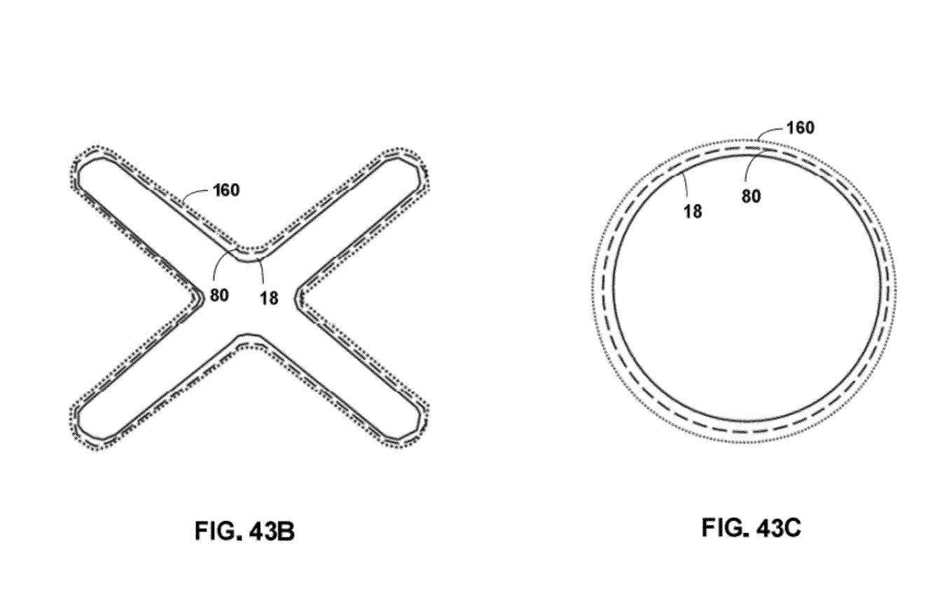

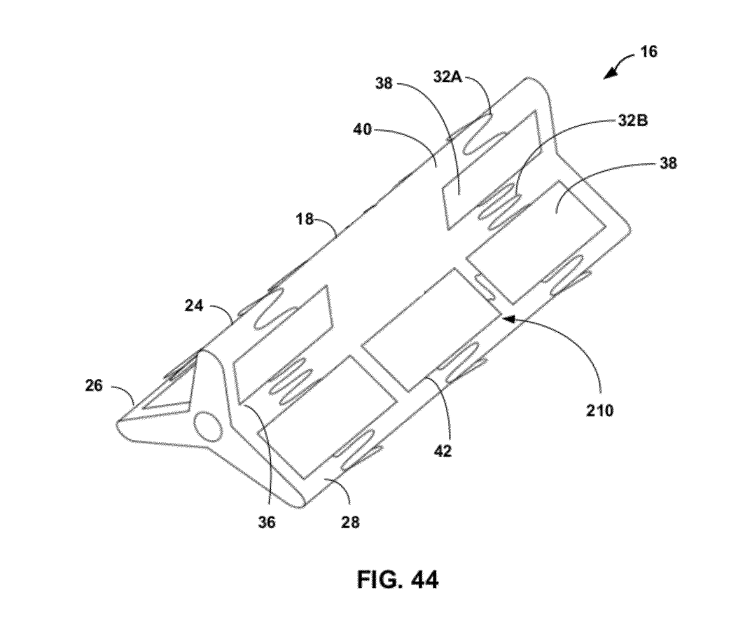

Stents in this disclosure can be made from typical materials, such as stainless steel, platinum enhanced stainless steel or cobalt chromium alloys, various polymers, magnesium, iron, or alloys thereof. One skilled in the art could utilize the various properties of potential stent materials to serve best as flexible connectors 32 or stiff elements 38 of the stent pattern such that the stent, e.g., stent 30, includes multiple materials around the circumference of the stent. Alternately, the stent could be a self-expanding stent that is held down by deformable rings, which are held down in the balloon folds, as shown and described below with respect to FIGS. 43A-C.

FIG. 43A is a flat plan view of an unwrapped self-expanding stent held down by deformable rings. In particular, FIG. 43A depicts an example stent, shown generally at 80, held down by one or more deformable rings 160A-D (“rings 160”). In the example shown in FIG. 43A, rings 160 are wrapped around the outer diameter of stent 80. In some examples, rings 160 are made of a plastically deformable alloy, e.g., stainless steel or the like. In one example, the rings are made of a deformable alloy such as platinum-iridium (Pt—Ir) to provide radiopacity.

FIG. 43B is a cross-sectional view of one example of a self-expanding stent held down by deformable rings. Stent 80 of FIG. 43B is constrained against wrapped balloon body 18 by one or more deformable rings 160. Because a self-expanding stent has an outward radial force in the constrained condition, rings 160 hold the stent down on the wrapped balloon body. FIG. 43C is a cross-sectional view of the self-expanding stent of FIG. 43B in an expanded state. It should noted that as an alternative to deformable rings 160, socks (or sox) may be used to keep the stent and balloon in a folded state. Socks are shown and described in detail in U.S. Pat. Nos. 6,607,552 and 6,478,814, the entire contents of each being incorporated herein by reference.

FIG. 44 is a perspective view of another example stent for use with a pleated balloon, in accordance an example of this disclosure. In particular, the stent of FIG. 44, shown generally at 210, has fewer elements 38 than stent 30 of FIG. 3A, for example. Such a stent can be used opposite a bifurcation, for example. A person of ordinary skill in the art will recognize that more elements 38 may be removed to create other geometries and configurations to be used inside of a vessel. For example, a C-shaped stent can be created.

It should be noted that the example stents described above can be circular stents that are crimped onto a shape such as shown in FIG. 2B. However, in other example implementations, the stents described in this disclosure are formed of polymeric materials which are already extruded in the shape shown in FIG. 3A, for example. In such an implementation, elements 38 are pre-positioned in the correct location so that an already formed balloon, e.g., balloon 16 of FIG. 2B, can be inserted into the stent shape.

As indicated above, stents implementing the techniques of this disclosure can include one or therapeutic agents disposed thereon, depending on the condition which is being treated. As used herein, the term “therapeutic agent includes genetic therapeutic agents, non-genetic therapeutic agents and cells. A drug may be used singly or in combination with other drugs. Drugs include genetic materials, non-genetic materials, and cells.

A therapeutic agent may be a drug or other pharmaceutical product such as non-genetic agents, genetic agents, cellular material, etc. Some examples of suitable non-genetic therapeutic agents include but are not limited to: antithrombogenic agents such as heparin, heparin derivatives, vascular cell growth promoters, growth factor inhibitors, etc. Where an agent includes a genetic therapeutic agent, such a genetic agent may include but is not limited to: DNA, RNA and their respective derivatives and/or components; hedgehog proteins, etc. Where a therapeutic agent includes cellular material, the cellular material may include but is not limited to: cells of human origin and/or non-human origin as well as their respective components and/or derivatives thereof.

Other active agents include, but are not limited to, antineoplastic, antiproliferative, antimitotic, antiinflammatory, antiplatelet, anticoagulant, antifibrin, antiproliferative, antibiotic, antioxidant, and antiallergic substances as well as combinations thereof.

Examples of antineoplastic/antiproliferative/antimitotic agents include, but are not limited to, paclitaxel (e.g., TAXOL® by Bristol-Myers Squibb Co., Stamford, Conn.), the olimus family of drugs including sirolimus (rapamycin), biolimus (derivative of sirolimus), everolimus (derivative of sirolimus), zotarolimus (derivative of sirolimus) and tacrolimus, methotrexate, azathiprine, vincristine, vinblastine, 5-fluorouracil, doxorubicin hydrochloride, mitomycin, cisplatin, vinblastine, vincristine, epothilones, endostatin, angiostatin and thymidine kinase inhibitors.

While the preventative and treatment properties of the foregoing therapeutic substances or agents are well-known to those of ordinary skill in the art, the substances or agents are provided by way of example and are not meant to be limiting. Other therapeutic substances are equally applicable for use with the disclosed methods and compositions.

Derivatives of many of the above mentioned compounds also exist which are employed as therapeutic agents and of course mixtures of therapeutic agents may also be employed.

For application, the therapeutic agent can be dissolved in a solvent or a cosolvent blend, and an excipient may also be added to the first coating composition.

Suitable solvents include, but are not limited to, dimethyl formamide (DMF), butyl acetate, ethyl acetate, tetrahydrofuran (THF), dichloromethane (DCM), acetone, acetonitrile, dimethyl sulfoxide (DMSO), butyl acetate, etc.

Suitable excipients include, but are not limited to, acetyl tri-n-butyl citrate (ATBC), acetyl triethyl citrate (ATEC), dimethyl tartarate (D, L, DL), diethyl tartarate (D, L, DL), dibutyl tartarate (D, L, DL), mono-, di- and tri-glycerol such as glycerol triacetate (triacetin), glycerol tributyrate (tributyrin), glycerol tricaprylate (tricarprin), sucrose octa acetate, glucose penta acetate (D, L, DL, and other C6 sugar variations), diethyl oxylate, diethyl malonate, diethyl maleate, diethyl succinate, dimethyl glutarate, diethyl glutarate, diethyl 3-hydroxy glutarate, ethyl gluconate (D, L, DL, and other C6 sugar variations), diethyl carbonate, ethylene carbonate, methyl acetoacetate, ethyl acetoacetate, butyl acetoacetate, methyl lactate, (D, L, or DL), dthyl lactate, (D, L, or DL), butyl lactate (D, L, or DL), methyl glycolate, ethyl glycolate, butyl glycolate, lactide (DD), lactide (LL), lactide (DL), glycolide, etc.

Suitable biodegradable polymeric excipients may include polylactide, polylactide-co-glycolide, polycaprolactone, etc. Other suitable polymeric excipients include, but are not limited to, block copolymers including styrenic block copolymers such as polystyrene-polyisobutylene-polystyrene triblock copolymer (SIBS), hydrogels such as polyethylene oxide, silicone rubber and/or any other suitable polymer material.

These lists are intended for illustrative purposes only, and not as a limitation on the scope of the present disclosure.

Various examples of the disclosure have been described. These and other examples are within the scope of the following claims.

1. A stent having a proximal end, a distal end, and a longitudinal axis, the stent comprising:
a plurality of first columns, the plurality of first columns extending from the proximal end to the distal end along the longitudinal axis, each of the respective first columns extending substantially parallel to the longitudinal axis, each of the respective first columns comprising a plurality of elements, each of the elements having a substantially polygonal shape;
a plurality of second columns, the plurality of second columns extending from the proximal end to the distal end along the longitudinal axis, each of the respective second columns extending substantially parallel to the longitudinal axis, each of the respective second columns between adjacent first columns, and each of the respective second columns comprising a plurality of first connectors, each of the first connectors having a curvilinear shape and extending in a substantially circumferential direction to connect adjacent first columns; and
a plurality of second connectors, wherein at least one of the second connectors extends between a first element and a second element.

2. The stent of claim 1, wherein the first element and the second element are longitudinally aligned along the longitudinal axis.

3. The stent of claim 1, wherein each of the plurality of elements define a solid structure.

4. The stent of claim 3, wherein at least one of the solid structures has at least one well region that defines at least one well, the at least one well having a depth.

5. The stent of claim 3, wherein the at least one of the well contains a therapeutic agent.

6. The stent of claim 1, wherein at least one of the plurality of elements defines a substantially hollow structure.

7. The stent of claim 1, wherein the polygonal shape is substantially quadrilateral.

8. The stent of claim 1, wherein the polygonal shape is substantially hexagonal.

9. The stent of claim 1, wherein the curvilinear shape is S-shaped.

10. The stent of claim 1, wherein the curvilinear shape is U-shaped.

11. The stent of claim 1, wherein the plurality of second connectors are arranged to form at least one substantially helical pathway along the stent.

12. The stent of claim 1, wherein the plurality of second connectors are arranged to form at least one pathway along the stent that is substantially parallel to the longitudinal axis.

13. The stent of claim 1, wherein the plurality of second connectors are arranged to form at least one staggered pathway along the stent.

14. The stent of claim 1, wherein the plurality of second connectors are substantially straight.

15. The stent of claim 14, wherein at least some of the plurality of second connectors are arranged in pairs.

16. The stent of claim 1, wherein the plurality of second connectors are curvilinear.

17. The stent of claim 1, wherein at least some of the plurality of second connectors extend between a first element in a first column and a second element in a second column.

18. A stent delivery system comprising:
a catheter comprising an expandable balloon, the balloon having an unpleated state and a pleated state, the balloon having at least two pleats in the pleated state, adjacent pleats being separated by a trough in the pleated state; and
a stent having a longitudinal axis, the stent comprising:
a plurality of interconnected annular bands, each annular band comprising:
a plurality of elements, each of the elements having a substantially polygonal shape; and
a plurality of first connectors, at least one of the first connectors extending between adjacent elements of an annular band, at least one of the first connectors being disposed adjacent at least one pleat of the balloon, and at least another of the first connectors being disposed adjacent at least one trough of the balloon; and
a plurality of second connectors, wherein adjacent annular bands are connected by at least one second connector.

19. The system of claim 18, wherein the at least one second connector extends between longitudinally aligned elements in adjacent annular bands.

20. The system of claim 18, wherein each of the plurality of elements define a solid structure.

21-33. (canceled)
